(19) United States
(12) Patent Application Publication
(10) Pub. No.: US 2019/0223758 A1
(43) Pub. Date: Jul. 25, 2019

(54) COMPOSITE PLANARITY MEMBER WITH INTEGRATED TRACKING SENSORS

(71) Applicant: St. Jude Medical International Holding S.à r.l., Luxembourg (LU)

(72) Inventor: Dale E. Just, Minneapolis, MN (US)

(21) Appl. No.: 16/327,587

(22) PCT Filed: Aug. 24, 2017
(86) PCT No.: PCT/IB2017/055109

§ 371 Date: Feb. 22, 2019

Related U.S. Application Data
(60) Provisional application No. 62/379,073, filed on Aug. 24, 2016.

(51) Int. Cl. A61B 5/06; A61M 25/01

(52) U.S. Cl. CPC A61B 5/065; A61M 25/0147; A61B 5/062

(57) ABSTRACT

A composite planarity member for a medical device that can comprise a planarity member and a tracking sensor coupled to the planarity member. A catheter that can comprise a tip electrode, a catheter body comprising a lumen, the catheter coupled to the tip electrode, and a composite planarity member comprising a planarity member and a flat coil coupled to the planarity member, wherein the composite planarity member is disposed within the lumen of the catheter body.

### BACKGROUND

### a. Field

The instant disclosure relates to planarity members with integrated tracking sensors. In one embodiment, the instant disclosure relates to planarity members with integrated tracking sensors for use in an MRI compatible, trackable ablation or diagnostic EP catheter.

### b. Background Art

Medical devices, catheters, and/or cardiovascular catheters, such as electrophysiology catheters are used in a variety of diagnostic and/or therapeutic medical procedures to diagnose and/or correct conditions such as atrial arrhythmias, including for example, ectopic atrial tachycardia, atrial fibrillation, and atrial flutter. Arrhythmias can create a variety of conditions including irregular heart rates, loss of synchronous atrioventricular contractions and stasis of blood flow in a chamber of a heart which can lead to a variety of symptomatic and asymptomatic ailments and even death.

A medical procedure in which an electrophysiology catheter is used includes a first diagnostic catheter deployed through a patient's vasculature to a patient's heart or a chamber or vein thereof. An electrophysiology catheter that carries one or more electrodes can be used for cardiac mapping or diagnosis, ablation and/or other therapy delivery modes, or both. Once at the intended site, treatment can include, for example, radio frequency (RF) ablation, cryoablation, laser ablation, chemical ablation, high-intensity focused ultrasound-based ablation, electroporation ablation or microwave ablation. An electrophysiology catheter imparts ablative energy to cardiac tissue to create one or more lesions in the cardiac tissue and oftentimes, a contiguous, and transmural lesion. This lesion disrupts undesirable cardiac activation pathways and thereby limits, corrals, or prevents errant conduction signals that can form or sustain arrhythmias.

To aid in the delivery of the medical device to the site, sensors (e.g., electrodes, electromagnetic coils) can be placed on the medical device, which can receive signals that are generated proximate to the patient from a device (e.g., electromagnetic field generator). Based on the received signals, an orientation and/or position of the medical device can be computed.

### BRIEF SUMMARY

The instant disclosure, in at least one embodiment, relates to a planarity member with integrated tracking sensors.

In one embodiment, a composite planarity member for a medical device can comprise a planarity member, and a tracking sensor coupled to the planarity member. In one embodiment, the planarity member can comprise a non-susceptible material. In another embodiment, the tracking sensor can comprise an electrical trace.

In another embodiment of the disclosure, a catheter can comprise a tip electrode, a catheter body can comprise a lumen, the catheter coupled to the tip electrode, and a composite planarity member can comprise a planarity member and a flat coil coupled to the planarity member. The composite planarity member can be disposed within the lumen of the catheter body. In one embodiment, the catheter can further comprise a lumen guide which can comprise a first channel and a second channel. In another embodiment, the second channel can be configured to secure the composite planarity member within a known location of the catheter body.

In yet another embodiment, a catheter can comprise a tip electrode, a catheter body comprising a lumen, the catheter coupled to the tip electrode, a steering mechanism coupled to a proximal end of the catheter body, wherein the steering mechanism is configured to deflect a distal portion of the catheter body, and a composite planarity member comprising a planarity member and a tracking sensor coupled to the planarity member, wherein the composite planarity member is disposed in the distal portion and within the lumen of the catheter body. The composite planarity member is configured to maintain the distal portion of the catheter shaft within a plane when the distal portion of the catheter shaft is deflected.

### BRIEF DESCRIPTION OF THE DRAWINGS

FIG. 1 depicts a diagrammatic view of an exemplary system for performing one or more diagnostic or therapeutic procedures, wherein the system comprises a magnetic field-based medical positioning system, in accordance with embodiments of the present disclosure.

FIG. 2 is an isometric, partial section view of one embodiment of a catheter comprising an electrode tip, a catheter body, and a composite planarity member.

FIG. 3 is a top down view of one embodiment of a composite planarity member.

FIG. 4 is a close up view of the distal portion of the composite planarity member illustrated in FIG. 3.

FIG. 5 is a detailed isometric view of a proximal coil disposed on one embodiment of a composite planarity member including conductor traces coupled to more distally located coils.

FIG. 6 is a is a close up view of the distal portion of another embodiment of a composite planarity member.

FIG. 7 is an isometric close-up view of a distal portion of another embodiment of a composite planarity member.

FIG. 8A is a close up view of a distal portion of another embodiment of a composite planarity member with a first trace layer deposited on a planarity member.

FIG. 8b is a close up view of a distal portion of the embodiment of a composite planarity member of FIG. 8A with a second trace layer deposited on the planarity member and the first trace layer.

FIG. 8C is a close up view of a distal portion of the embodiment of a composite planarity member of FIGS. 8A and 8B with a third trace layer deposited on the planarity member and the first and second trace layers.

FIG. 9A is a top down view of another embodiment of a composite planarity member.

FIG. 9B is an isometric, close up view of the distal portion of the composite planarity member illustrated in FIG. 9A.

FIG. 9C is a close up view of a distal portion of the embodiment of a composite planarity member of FIGS. 9A and 9B with a first trace layer deposited on a planarity member.

FIG. 9D is a close up view of a distal portion of the embodiment of a composite planarity member of FIGS. 9A-9C with a second trace layer deposited on the planarity member.

FIG. 9E is a top down, close up view of the distal portion of the composite planarity member illustrated in FIGS. 9A-9D.

### DETAILED DESCRIPTION

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

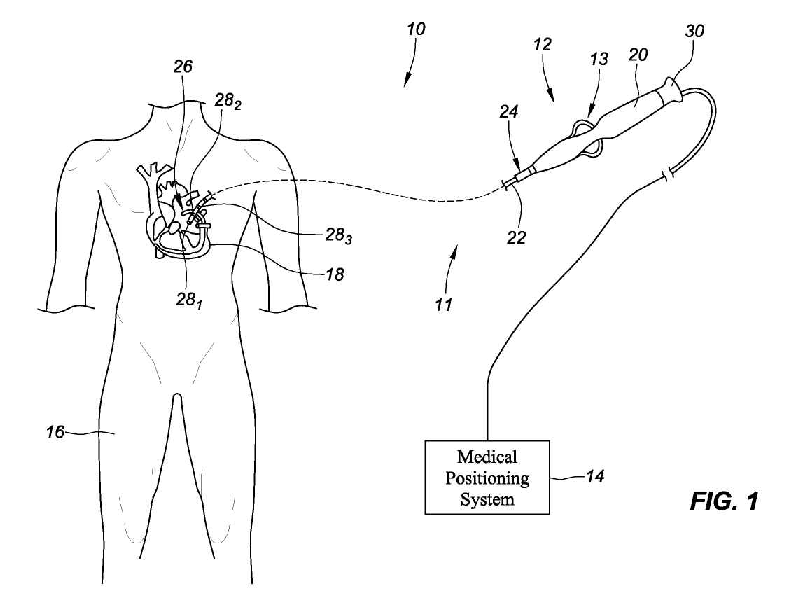

In some embodiments, and with reference to FIG. 1, the system 10 can include a medical device 11 and a medical positioning system 14. The medical device 11 can include an elongate medical device such as, for example, a catheter or a sheath. For purposes of illustration and clarity, the description below will be limited to an embodiment wherein the medical device 11 comprises a catheter (e.g., catheter 12). It will be appreciated, however, that the present disclosure is not meant to be limited to such an embodiment, but rather in other exemplary embodiments, the medical device may comprise other elongate medical devices, such as, for example and without limitation, sheaths, introducers and the like. Accordingly, as used in various embodiments herein, a “catheter” includes any elongated structure that can be inserted into and/or through a body cavity, duct, and/or vessel. In at least one embodiment, a catheter may be hollow, such as an introducer or coronary catheter, and/or define a lumen therethrough for passing fluid or another medical device, such as a guidewire or another catheter, for example. However, in various embodiments, a catheter may be closed at least at its distal end, such as an electrophysiology catheter or guidewire.

With continued reference to FIG. 1, the catheter 12 can be configured to be inserted into a patient's body 16, and more particularly, into the patient's heart 18. The catheter 12 may include a handle 20 that has a proximal end 30, a steering mechanism 13, a shaft 22 having a proximal end portion 24 and a distal end portion 26, and one or more sensors 28 mounted in or on the shaft 22 of the catheter 12. The steering mechanism can be coupled to a proximal end of the catheter body. The steering mechanism 13 can be configured to deflect a distal portion of the catheter body As used herein, “sensor 28” or “sensors 28” may refer to one or more sensors 28₁, 28₂, . . . 28_{N}, as appropriate and as generally depicted. In an exemplary embodiment, the sensors 28 are disposed at the distal end portion 26 of the shaft 22. The catheter 12 may further include other conventional components such as, for example and without limitation, a temperature sensor, additional sensors or electrodes, ablation elements (e.g., ablation tip electrodes for delivering RF ablative energy, high intensity focused ultrasound ablation elements, etc.), and corresponding conductors or leads.

The shaft 22 can be an elongate, tubular, flexible member configured for movement within the body 16. The shaft 22 supports, for example and without limitation, sensors and/or electrodes mounted thereon, such as, for example, the sensors 28, associated conductors, and possibly additional electronics used for signal processing and conditioning. The shaft 22 may also permit transport, delivery, and/or removal of fluids (including irrigation fluids, cryogenic ablation fluids, and bodily fluids), medicines, and/or surgical tools or instruments. The shaft 22 may be made from conventional materials such as polyurethane, and define one or more lumens configured to house and/or transport electrical conductors, fluids, or surgical tools. The shaft 22 may be introduced into a blood vessel or other structure within the body 16 through a conventional introducer. The shaft 22 may then be steered or guided through the body 16 to a desired location, such as the heart 18, using means well known in the art.

The sensors 28 mounted in or on the shaft 22 of the catheter 12 may be provided for a variety of diagnostic and therapeutic purposes including, for example and without limitation, electrophysiological studies, pacing, cardiac mapping, and ablation. In an exemplary embodiment, one or more of the sensors 28 are provided to perform a location or position sensing function. More particularly, and as will be described in greater detail below, one or more of the sensors 28 are configured to be a positioning sensor that provides information relating to the location (e.g., position and orientation) of the catheter 12, and the distal end portion 26 of the shaft 22 thereof, in particular, at certain points in time. Accordingly, in such an embodiment, as the catheter 12 is moved along a surface of a structure of interest of the heart 18 and/or about the interior of the structure, the sensor(s) 28 can be used to collect location data points that correspond to the surface of, and/or other locations within, the structure of interest. These location data points can then be used for a number of purposes such as, for example and without limitation, the construction of surface models of the structure of interest. The system 14 may include various visualization, mapping and navigation components as known in the art, including, for example, an EnSite™ Velocity™ system commercially available from St. Jude Medical, Inc., or as seen generally, for example, by reference to U.S. Pat. No. 7,263,397, or 7,885,707, both of which are hereby incorporated by reference in their entireties as though fully set forth herein. The system 14 can further be used with a magnetic resonance tracking system. The magnetic resonance tracking system can use the magnetic resonance signals from each sensor to determine the three-dimensional coordinates of each sensor. With reference to the present disclosure, the system 14 is configured to, among other things, collect cardiologic data, particularly impedance and temperature information, from intra-tip electrodes and sensors, respectively, mounted to the medical device to thereby provide accurate, reliable and complimentary lesion information without regard to the orientation of the medical device.

For purposes of clarity and illustration, the description below will be with respect to an embodiment wherein a single sensor 28 of the catheter 12 comprises a positioning sensor. It will be appreciated, however, that in other exemplary embodiments, which remain within the spirit and scope of the present disclosure, the catheter 12 may comprise more than one positioning sensor as well as other sensors or electrodes configured to perform other diagnostic and/or therapeutic functions. As will be described in greater detail below, the sensor 28 can include a pair of leads extending from a sensing element thereof (e.g., a coil) that are configured to electrically couple the sensor 28 to other components of the system 10, such as, for example, the medical positioning system 14. In some embodiments, the sensing element can be an electromagnetic position sensor, such as a wound coil, which can sense a magnetic field that is generated in proximity to the patient. Depending on a position and orientation (P&O) of the electromagnetic position sensor, different electrical signals can be generated by the coil and transferred to the medical positioning system, for a determination of a location reading that can be indicative of the P&O of the electromagnetic position sensor.

The location readings may each include at least one or both of a position and an orientation (P&O) relative to a reference coordinate system, which may be the coordinate system of medical positioning system 14. For some types of sensors, the P&O may be expressed with five degrees-of-freedom (five DOF) as a three-dimensional (3D) position (i.e., a coordinate in three axes X, Y and Z) and two-dimensional (2D) orientation (e.g., an azimuth and elevation) of sensor 28 in a magnetic field relative to a magnetic field generator(s) or transmitter(s) and/or a plurality of electrodes in an applied electrical field relative to an electrical field generator (e.g., a set of electrode patches). For other sensor types, the P&O may be expressed with six degrees-of-freedom (six DOF) as a 3D position (i.e., X, Y, Z coordinates) and 3D orientation (i.e., roll, pitch, and yaw).

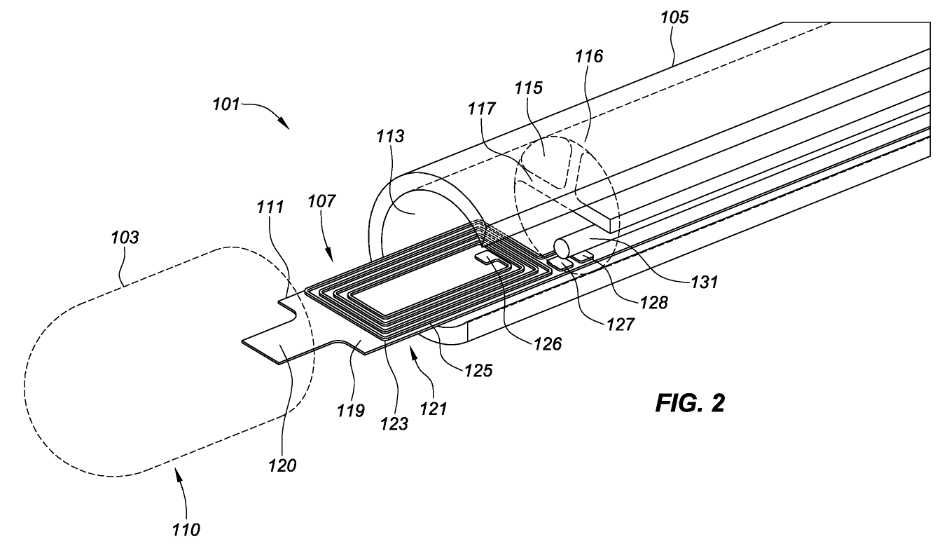

FIG. 2 illustrates an isometric, partial section view of one embodiment of a catheter 101. The catheter 101 can comprise a tip electrode 103, a catheter body 105, a lumen guide 115, and a composite planarity member 107. The composite planarity member 107 can extend along at least a portion of the length of the catheter in order to aid the catheter in deflection along a single axis. The composite planarity member 107 can be configured to maintain the planarity of a deflectable catheter shaft section as the deflectable catheter shaft section deflects. The catheter body 105 can be deflected by a steering mechanism coupled to a proximal end of the catheter body 105. The steering mechanism can be coupled to at least one pull wire (not shown) that can extend through the catheter body. The at least one pull wire can be coupled to a distal portion of the catheter body. In one embodiment, the at least one pull wire can be coupled to the composite planarity member. In another embodiment, the at least one pull wire can be coupled to a pull ring coupled to a distal portion of the catheter body. In other embodiments, the steering mechanism can be configured to deflect a distal portion of the catheter as would be known to one of ordinary skill in the art. The tip electrode 103 can comprise a tip electrode distal end 110 and a tip electrode proximal end 111. The tip electrode distal end 110 can impart energy to a target tissue. The tip electrode proximal end 111 can be sized and configured to fit within a distal end of the catheter body 105 and can further comprise a hollow portion (not shown) sized and configured to couple to a distal end of the composite planarity member 107. The catheter body 105 can comprise a lumen 113 extending from a proximal end to a distal end of the catheter body 105. In other embodiments, the catheter can further comprise at least one ring electrode. The at least one ring electrode can be coupled to the catheter shaft in a location proximal the tip electrode. The at least one ring electrode can be configured to sense electrical information.

The lumen guide 115 can fit within the lumen 113 The lumen guide 115 can comprise a first channel 116 and a second channel 117. In the illustrated embodiment, the first channel 116 can comprise a cavity extending from a proximal end of the lumen guide 115 to a distal end of the lumen guide 115. The first channel 116 can allow for wires, conductors, or other components to extend from a proximal end of the catheter body 105 to a portion of the catheter 101 located more distal than a distal end of the lumen guide 115. In other embodiments, the first channel can allow for wires, conductors, or other components to exit the catheter body 105 between the proximal end and the distal end of the catheter body 105. One non-limiting example is a wire electrically connected to a ring electrode that is coupled to an outer surface of the catheter body 105. The second channel 117 can comprise a cavity sized and configured to support a proximal portion of the composite planarity member 107. When the composite planarity member 107 is positioned within the second channel 117, the composite planarity member 107 can be secured in a known location and resistant to movement within the catheter body 105. In one embodiment, the second channel 117 can extend from the distal end of the lumen guide 115 to a position between the distal end and the proximal end of the lumen guide 115. In this embodiment, the second channel 117 does not extend through the entirety of the lumen guide 115.

The composite planarity member 107 can comprise a planarity member 119, a sensor 121, and a planarity distal projection 120. The planarity member 119 can comprise a substrate that can accept electrical traces. In some embodiments, the substrate can be formed of a semi-rigid material that can be flat (e.g., planar) and/or can include a flat surface, upon which the sensor can be disposed. The substrate can be formed as, for example, a planar rectangle, square, circle, ellipses, or other shape. In the illustrated embodiment, the planarity member 119 is formed as an elongated, planar, rectangular component comprising a planarity distal projection 120 at a distal end. The planarity distal projection 120 can be sized and configured to fit within the tip electrode 103 or other feature of the catheter to secure the planarity member 119 and the composite planarity member 107 within the catheter 101. The substrate can comprise polymers, polyesters, polyimides, glass, and various adhesives. Further substrates as would be known to one of ordinary skill in the art can be used to accept an electrical trace and impart a desired rigidity to the planarity member. In one embodiment, the planarity member can comprise materials that can safely operate in an MRI environment as a non-susceptible replacement to a metallic planarity beam. He materials used to achieve a non-susceptibility to an MRI environment include those listed above as well as other materials known to one of ordinary skill in the art. In one embodiment, the composite planarity member can comprise a printed circuit board.

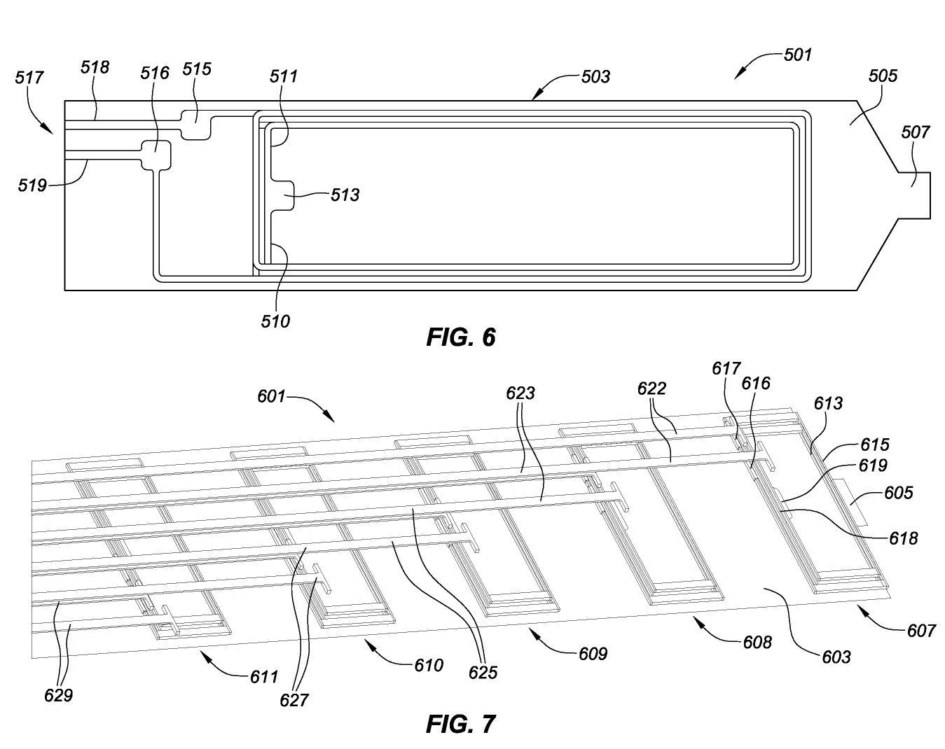

The sensor 121 can comprise a flat sensor either coupled to or deposited on the planarity member. The sensor 121 can be configured to be a positioning sensor that provides information relating to the location (e.g., position and orientation) of the catheter 12, and the distal end portion 26 of the shaft 22 thereof, in particular, at certain points in time. Accordingly, in such an embodiment, as the catheter 12 is moved along a surface of a structure of interest of the heart 18 and/or about the interior of the structure, the sensor(s) 28 can be used to collect location data points that correspond to the surface of, and/or other locations within, the structure of interest. These location data points can then be used for a number of purposes such as, for example and without limitation, the construction of surface models of the structure of interest. As discussed above, the sensor can be an electromagnetic sensor, a magnetic resonance sensor, or another sensor as would be known to one of ordinary skill in the art. In one embodiment, the sensor can comprise a thickness less than the planarity member it is coupled to. In another embodiment, the sensor can be integrally formed with the planarity member. In yet another embodiment, the sensor can be integrally formed and completely surrounded by the planarity member. In the illustrated embodiment, the sensor 121 can comprise a first trace layer 123, a second trace layer 125, a trace layer connection 126, a first conductor connection 127, and a second conductor connection 128. In some embodiments, the sensor 121 can be arranged in an elongated pattern that aligns with a longitudinal axis of the planarity member 119. In one embodiment, the sensor can be a planar elongated coil and can extend further in a first direction (e.g., along the longitudinal axis of the planarity member) than in a second direction (e.g., along an axis orthogonal to the longitudinal axis of the planarity member). In other embodiments, the sensor can extend further in a second direction (e.g., along an axis orthogonal to the longitudinal axis of the planarity member) than in a first direction (e.g., along the longitudinal axis of the planarity member). One embodiment of a sensor of this nature is illustrated and further described in FIG. 7. The first trace layer 123, the second trace layer 125, the trace layer connection 126, the first conductor connection 127, and the second conductor connection 128 can be formed from a conductive trace that can be formed by thin film deposition. The conductive trace may also be formed using a conductive ink through an ink jet process or additive manufacturing process. Additive manufacturing processes include material jetting and binder jetting and others described in American Society for Testing Materials (ASTM) group 42 definitions. The traces can then be covered with additional coatings. For example, a non-conductive or dielectric coating may be applied on top of the conductive trace for protection. Other embodiments of a sensor according to the disclosure can comprise various other shapes and/or patterns (e.g. an oval pattern, a rectangular pattern, or an elliptical pattern). The sensor 121 can have a gap or a spacing between the adjacent portions of the trace layers that is equal throughout the sensor. By placing the sensors at known locations on the composite planarity member, the position of the sensors can be controlled during manufacturing and, as a result, tracking of the device can be achieved with more reliable results for a lower cost of manufacture.

In one embodiment, the first sensor 131 can comprise a magnetic sensor disposed in a location that is off a longitudinal axis of the catheter 101. By placing the first sensor 131 in an off-axis location within the catheter body 105 and in a known location relative to other location sensors, a location of the catheter with six degrees of freedom can be determined.

In another embodiment, the sensor can be formed from a conductive wire segment. The conductive wire segment can be concentrically wound around an elongated central origin that extends along a longitudinal axis. The conductive wire segment can be concentrically wound in an elongated pattern (e.g. an oval pattern, a rectangular pattern, or an elliptical pattern). The sensor can be shaped in a generally flat (e.g. planar) configuration to fit on a planarity member.

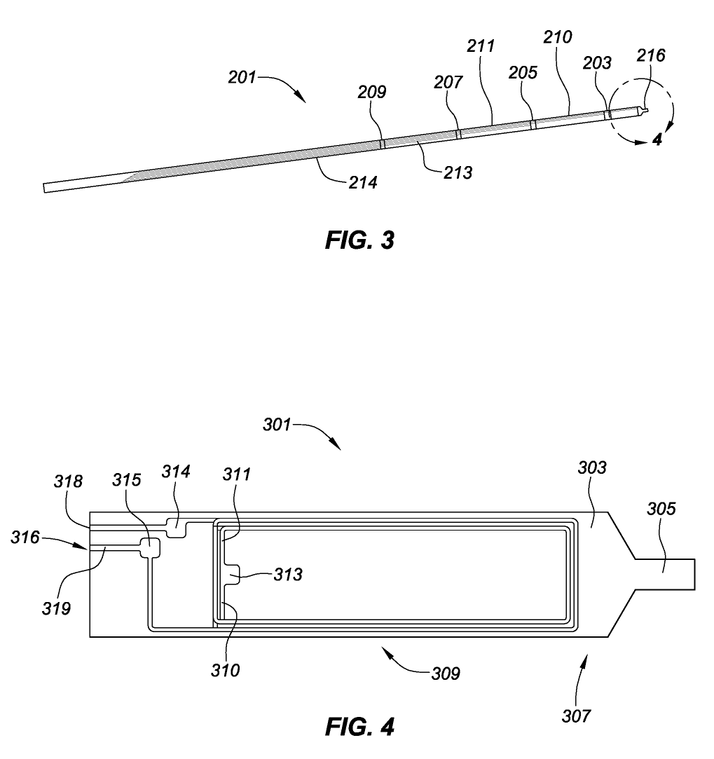

FIG. 3 illustrates a top down view of a composite planarity member 201. The composite planarity member 201 can comprise a first coil 203, a second coil 205, a third coil 207, a fourth coil 209, a first conductor trace set 210, a second conductor trace set 211, a third conductor trace set 213, a fourth conductor trace set 214, a planarity member 215, and a planarity distal projection 216. The plurality of conductor trace sets can each extend from an individual coil to a proximal end of the composite planarity member. Each of the plurality of conductor trace sets can be electrically coupled to another device to transfer signals to or from an individual coil. In some embodiments, one or more of the conductor trace sets can terminate before a distal end of the composite planarity member.

FIG. 4 illustrates a close up view of the distal end 307 of the composite planarity member 301 depicted in FIG. 3. The composite planarity member 301 comprises the planarity member 303, the planarity distal projection 305, and the first coil 309. The first coil 309 comprises a first coil trace 310, a second coil trace 311, a coil connection pad 313, a first trace conductor pad 314, a second trace conductor pad 315, and a first conductor trace set 316. The first conductor trace set can further comprise a first conductor trace 318 and a second conductor trace 319. The second coil trace 311 can overlay the first coil trace 310 on the planarity member 303.

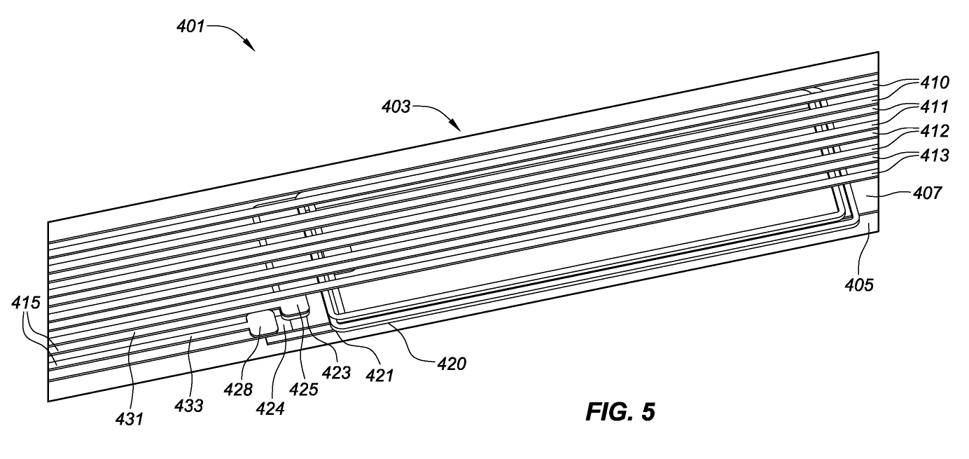

FIG. 5 depicts an isometric view of a portion of one embodiment of a composite planarity member 401. The close-up view of the composite planarity member 401 depicted in FIG. 5 is located in a proximal portion of the composite planarity member to those seen previously. The composite planarity member 401 can comprise a first planarity board 405, a second planarity board 407, a coil 403, a first conductor trace set 410, a second conductor trace set 411, a third conductor trace set 412, a fourth conductor trace set 413, and a fifth conductor trace set 415. In one embodiment, the illustrated coil 403 can comprise a fifth coil. Each of the conductor trace sets can couple to a component or sensor located on a more distal portion of the composite planarity member 401. In one embodiment, each of the conductor trace sets can couple to a coil disposed on the composite planarity member. The coil 403 can comprise a first coil trace 420, a second coil trace 421, a first coil trace pad 423, and a second coil trace pad 424. The fifth conductor trace set 415 can comprise a ninth conductor trace 431, a tenth conductor trace 433, a first conductor trace pad 427, and a second conductor trace pad 428. The first conductor trace pad 42 can be electrically coupled to the ninth conductor trace 431 and the second conductor trace pad 428 can be electrically coupled to the tenth conductor trace 433. The first conductor trace pad 427 can be electrically coupled to the first coil trace pad 423 and the second conductor trace pad 428 can be electrically coupled to the second coil trace pad 424. In the illustrated embodiment, the plurality of conductor trace sets can be deposited on a second planarity board 407. The first coil trace 420 and the second coil trace 421 can be deposited on the first planarity board 405. In one embodiment, the first conductor trace pad 427 can be coupled to the first coil trace pad 423 through a micro-via that extends through the second planarity board 407. Further, the second conductor trace pad 428 can be coupled to the second coil trace pad 424 through another micro-via that passes through the second planarity board 407.

FIG. 6 depicts a top down view of another embodiment of a composite planarity member 501 according to the disclosure. The composite planarity member 501 comprises a planarity member 505, a planarity distal projection 507, and a first coil 503. The first coil 503 comprises a first coil trace 510, a second coil trace 511, a coil connection pad 513, a first trace conductor pad 515, a second trace conductor pad 516, and a first conductor trace set 517. The first conductor trace set 517 can further comprise a first conductor trace 518 and a second conductor trace 519.

FIG. 7 illustrates an isometric view of another embodiment of a composite planarity member 601. The illustrated composite planarity member 601 comprises a planarity member 603, a first coil 607, a second coil 608, a third coil 609, a fourth coil 610, a fifth coil 611, a first conductor trace set 621, a second conductor trace set 623, a third conductor trace set 625, a fourth conductor trace set 627, and a fifth conductor trace set 629. Each of the coils illustrated in FIG. 7 can extend further in a second direction (e.g., along an axis orthogonal to a longitudinal axis of the planarity member) than in a first direction (e.g., along the longitudinal axis of the planarity member). The planarity member 603 can comprise a planarity distal projection 605. The first coil 607 can comprise a first coil trace 613, a second coil trace 615, a first coil connection pad 618, a second coil connection pad 619, a first coil trace pad 616, and a second coil trace pad 617. Each of the other coils depicted in the figure can comprise similar corresponding elements. As described above, the first coil trace 613 and the second coil trace 615 can each comprise at least one rectangular shaped coil and the first coil trace 613 can be electrically coupled to the second coil trace 615 through a micro-via electrically coupling the first coil connection pad 618 to the second coil connection pad 619.

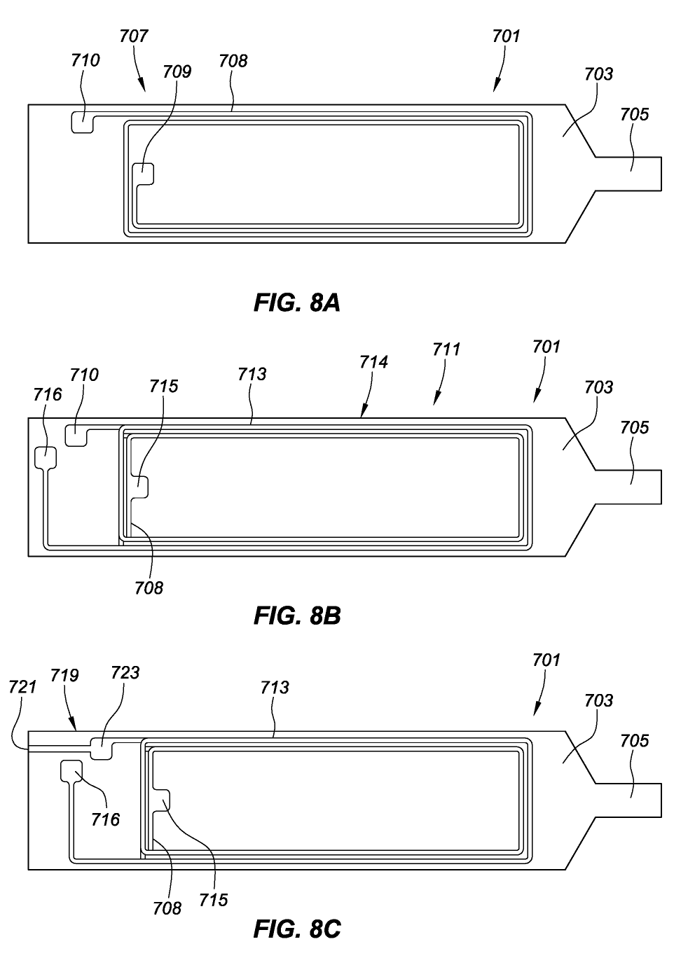

FIGS. 8A-8C illustrate a top down view of the multiple trace layers that can be used to form a coil as disclosed herein. FIG. 8A illustrates a composite planarity member 701 comprising a planarity member 703, a planarity distal projection 705, and a first trace layer 707. The first trace layer 707 is deposited on the planarity member 703 and can comprise a first coil trace 708, a first coil connection pad 709, and a first trace layer conductor termination 710. In the illustrated embodiment, the first coil trace 708 can comprise two longitudinally extending rectangularly shaped portions. The inner rectangularly shaped portion can be inset within the outer rectangularly shaped portion and the first coil connection pad 709 can be disposed in an internal portion of the inner rectangularly shaped portion. In other embodiments, the first coil trace can comprise more or less concentrically formed shapes. As illustrated the first coil trace 708 can comprise an unbroken trace extending in a distal direction from the first trace layer conductor termination 710 to the first coil connection pad 709.

FIG. 8B depicts the composite planarity member 701 of FIG. 8A with the addition of a second trace layer 714 to the composite planarity member 701. The second trace layer 714 can be deposited on the planarity member 703 on top of the first trace layer 708 to form a coil 711. The second trace layer 714 can comprise a second coil trace 713, a second coil connection pad 715, and a second trace layer conductor termination 716. In the illustrated embodiment, the second coil trace 713 can comprise two longitudinally extending rectangularly shaped portions. The inner rectangularly shaped portion can be inset within the outer rectangularly shaped portion and the second coil connection pad 715 can be disposed in an internal portion of the inner rectangularly shaped portion. In other embodiments, the second coil trace can comprise more or less concentrically formed shapes. The second coil connection pad 715 can be disposed above the first coil connection pad 709 and a plated micro-via can be used to electrically couple the first coil connection pad 709 to the second coil connection pad 715. By coupling the first coil trace 708 to the second coil trace 713, an electrical signal can be transmitted to or from the coil 711 by connecting the first trace layer conductor termination 710 and the second trace layer conductor termination 716 to a system as described above.

FIG. 8C depicts the composite planarity member 701 of FIG. 8B with the addition of a third trace layer 719 to the composite planarity member 701. The third trace layer 719 can be deposited on the planarity member 703 on top of the second trace layer 714 to electrically couple the coil 711 to a component located proximal of the coil 711. The third trace layer 719 can comprise a first conductor trace pad 723 and a first conductor trace 721. The first conductor trace pad 723 can be electrically coupled to the first trace layer conductor termination 710 by a plated micro-via and the first conductor trace pad 723 can be electrically coupled to the first conductor trace 721. Further, the composite planarity member 701 can comprise a fourth trace layer comprising a second conductor trace pad (not shown) and a second conductor trace (not shown). The second conductor trace pad can be electrically coupled to the second trace layer conductor termination 716 by a plated micro-via and the second conductor trace pad can be coupled to the second conductor trace.

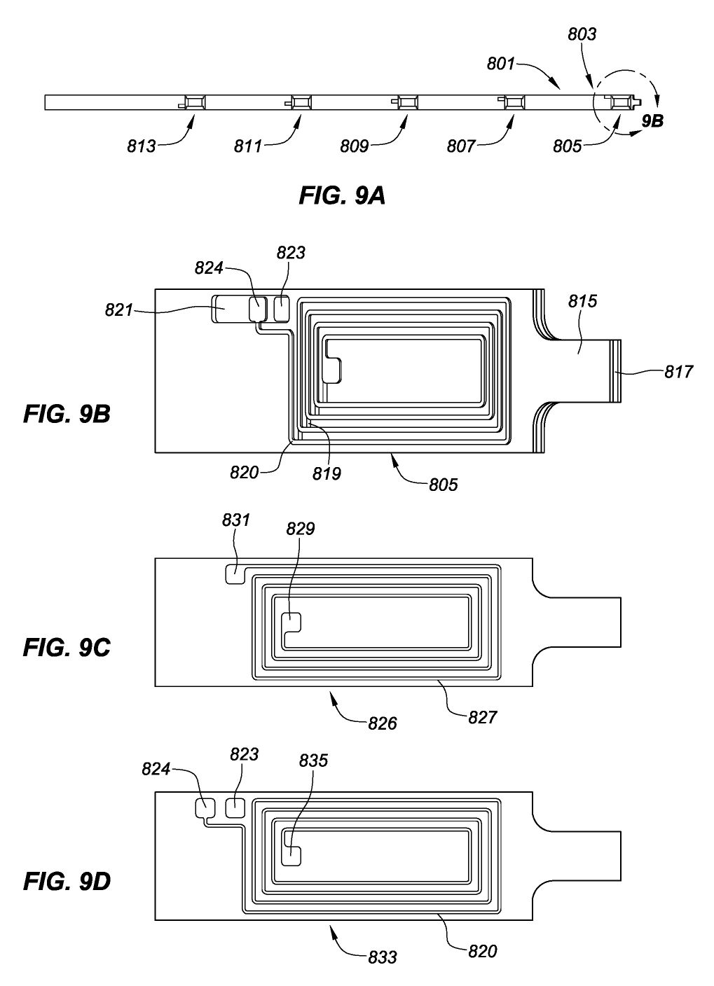

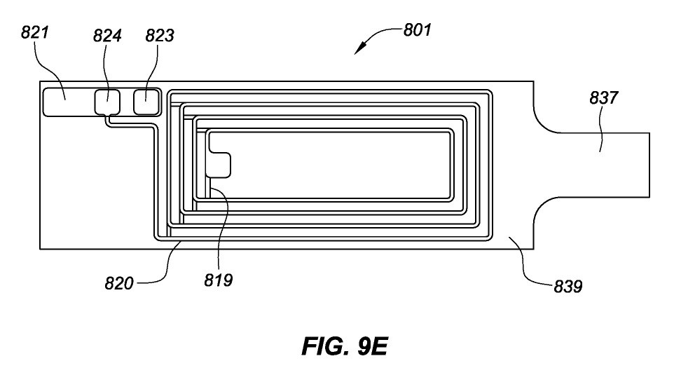

FIG. 9A illustrates a top down view of another embodiment of a composite planarity member 801. As discussed below, the embodiment comprises a stiffener layer with a window for the connection of conductors to the coils disposed on the composite planarity member 801. The composite planarity member 801 can comprise a first coil 805, a second coil 807, a third coil 809, a fourth coil 811, and a fifth coil 813. FIG. 9B illustrates a close-up view of the distal end 803 of the composite planarity member 801 depicted in FIG. 9A. The composite planarity member 801 can further comprise a stiffener layer 815, a planarity member 817, a window 821, a first coil trace pad 823, a second coil trace pad 824 a first coil trace 819, and a second coil trace 820. The stiffener layer 815 can comprise a material that can enhance the stiffness of the composite planarity member 801. When coupled, the stiffener layer 815 and the planarity member 817 comprise a compound planarity member. FIG. 9C illustrates a top down view of a portion of the planarity member 817 of FIG. 9B. The planarity member 817 further comprises a first trace layer 826 deposited thereon. The first trace layer 826 comprises a first coil trace 827, a first coil connection pad 829, and a first trace layer conductor termination 831. In the illustrated embodiment, the first coil trace 827 can comprise four longitudinally extending rectangularly shaped inset traces with the first coil connection pad 829 disposed in an internal portion of the first coil trace 827. FIG. 9D illustrates a top down view of a portion of the planarity member 817 of FIG. 9B. The planarity member 817 further comprises a second trace layer 833 deposited thereon. The second trace layer 833 comprises a second coil trace 820, a second coil connection pad 835, the first coil trace pad 823, and the second coil trace pad 824. The first coil trace pad 823 can be electrically coupled to the first layer conductor termination 831 through a micro-via. The electrical connection between the first coil trace pad 823 and the first layer conductor termination 831 can allow for a conductor to be electrically coupled to the first trace layer 826 through the window 821 in the stiffener layer 815. In the illustrated embodiment, the second coil trace 820 can comprise four longitudinally extending rectangularly shaped inset traces with the second coil connection pad 835 disposed in an internal portion of the second coil trace 820. FIG. 9E depicts a top down view of a distal portion of the composite planarity member 801. The composite planarity member 801 can further comprise a composite distal projection 837, a compound planarity member 839, a window 821, a first coil trace pad 823, a second coil trace pad 824, a first coil trace 819, and a second coil trace 820.

What is claimed is:

1. A composite planarity member for a medical device comprising:
a planarity member;
and a tracking sensor coupled to the planarity member.

2. The composite planarity member for a medical device according to claim 1, wherein the tracking sensor comprises a flat coil.

3. The composite planarity member for a medical device according to claim 2, wherein the flat coil comprises an electrical trace.

4. The composite planarity member for a medical device according to claim 3, wherein the coil further comprises a first trace layer and a second trace layer.

5. The composite planarity member for a medical device according to claim 4, wherein the flat coil further comprises a micro-via and wherein the micro-via electrically couples the first trace layer to the second trace layer.

6. The composite planarity member for a medical device according to claim 5, wherein the coil further comprises a first coil connection pad and a second coil connection pad and wherein the micro-via is disposed between the first coil connection pad and the second coil connection pad.

7. The composite planarity member for a medical device according to claim 4, wherein the second trace layer overlays the first trace layer.

8. The composite planarity member for a medical device according to claim 4, wherein the first trace layer comprises at least two rectangularly shaped portions and wherein the second trace layer comprises at least two rectangularly shaped portions.

9. The composite planarity member for a medical device according to claim 8, further comprising a stiffener layer coupled to the planarity member.

10. The composite planarity member for a medical device according to claim 9, wherein the stiffener layer comprises a window configured to enable a pair of conductive members to couple to the coil.

11. The composite planarity member for a medical device according to claim 4, further comprising a third trace layer, wherein the third trace layer is configured to electrically couple the coil to a proximal end of the composite planarity member,

12. The composite planarity member for a medical device according to claim 1, wherein the planarity member comprises a non-susceptible material.

13. A catheter comprising:
a tip electrode;
a catheter body comprising a lumen, the catheter coupled to the tip electrode; and
a composite planarity member comprising a planarity member and a flat coil coupled to the planarity member, wherein the composite planarity member is disposed within the lumen of the catheter body.

14. The catheter according to claim 13, further comprising a sensor disposed within the lumen of the catheter body.

15. The catheter according to claim 14, wherein the sensor is disposed off a longitudinal axis of the catheter body.

16. The catheter according to claim 15, wherein the sensor is a coil.

17. The catheter according to claim 13, further comprising at least one ring electrode coupled to an outer surface of the catheter body.

18. The catheter according to claim 13, wherein the tracking sensor comprises a flat coil.

19. A catheter comprising:
a tip electrode;
a catheter body comprising a lumen, the catheter coupled to the tip electrode;
a steering mechanism coupled to a proximal end of the catheter body, wherein the steering mechanism is configured to deflect a distal portion of the catheter body; and
a composite planarity member comprising a planarity member and a tracking sensor coupled to the planarity member, wherein the composite planarity member is disposed in the distal portion and within the lumen of the catheter body,
wherein the composite planarity member is configured to maintain the distal portion of the catheter shaft within a plane when the distal portion of the catheter shaft is deflected.

20. The catheter according to claim 19, wherein the tracking sensor comprises a flat coil.
